(19) United States
(12) Patent Application Publication
(10) Pub. No.: US 2012/0095324 A1
(43) Pub. Date: Apr. 19, 2012

(54) METHOD FOR A NUCLEAR MEDICINE EXAMINATION

(75) Inventor: Sebastian Schmidt, Weisendorf (DE)

(73) Assignee: SIEMENS AKTIENGESELLSCHAFT, Munich (DE)

(21) Appl. No.: 13/272,761

(22) Filed: Oct. 13, 2011

(30) Foreign Application Priority Data
Oct. 15, 2010 (DE) 10 2010 42 506.0

(51) Int. Cl. A61B 6/00; A61B 6/03; A61B 5/055; A61B 6/02; A61K 51/04; A61K 51/02

(52) U.S. Cl. 600/411; 424/1.73; 424/1.65; 424/1.61

(57) ABSTRACT

A method for a nuclear medicine examination of a patient is disclosed. In at least one embodiment of the method, a magnetic resonance recording of an examination region of the patient is created after a magnetic resonance contrast agent has been administered to the patient. A distribution of the magnetic resonance contrast agent in the examination region is automatically determined from the magnetic resonance recording. After a nuclear medicine tracer has been administered to the patient, a nuclear medicine recording of the examination region of the patient is created. The magnetic resonance contrast agent and the nuclear medicine tracer have essentially identical pharmacokinetic properties. The nuclear medicine recording is corrected as a function of the distribution of the magnetic resonance contrast agent in the examination region.

### PRIORITY STATEMENT

The present application hereby claims priority under 35 U.S.C. §119 on German patent application number DE 10 2010 042 506.0 filed Oct. 15, 2010, the entire contents of which are hereby incorporated herein by reference.

### FIELD

At least one embodiment of the present invention generally relates to a method for a nuclear medicine examination of a patient with the aid of a magnetic resonance recording and a nuclear medicine recording, with a magnetic resonance contrast agent and a nuclear medicine tracer being used, which have essentially identical pharmacokinetic properties. At least one embodiment of the invention further relates to the use of magnetic resonance contrast agents and nuclear medicine tracers with essentially identical pharmacokinetic properties, which are used during an imaging examination of a patient, with a nuclear medicine recording being corrected as a function of information from a magnetic resonance recording.

### BACKGROUND

During examinations of the brain using imaging methods, contrast agents or radio tracers are usually administered so as to be able to better assess lesions, for instance a brain tumor. A distribution of the contrast agent or of the radio tracer in the brain is shown in the image information thus obtained.

The assessment is however hindered in that the contrast agent or the radio tracer has to overcome the blood-brain barrier, and the contrast agent or the radio tracer is specifically absorbed by the lesion and/or accumulates specifically therein. Here an absorption and/or accumulation of the contrast agent or of the tracer is/are dependent on overcoming the blood-brain barrier. If the contrast agent or the tracer overcomes the blood-brain barrier less effectively for instance, the contrast agent or the tracer is only marginally absorbed or not at all. This may result in inaccurate assessments. For instance, a so-called “pseudo progression” may occur if in the case of a patient undergoing a combined radiochemotherapy, a significant malfunction of the blood-brain barrier results and contrast agent is increasingly able to penetrate the brain. As a result, a brain tumor may appear much larger than it actually is. Conversely, contrast agents or tracers may show too low a level of accumulation for instance if they are less able to overcome the blood-brain barrier.

For body regions outside of the head, a method for combining positron emission tomography information (PET) with magnetic resonance perfusion and diffusion information (MR) is known from U.S. Pat. No. 7,482,592. With the method, a positron emission measurement is implemented using a marker substance in a body region of an examination object to be examined in order to determine positron emission measurement information. At the same time, image recordings of the body region to be examined are created by way of a second medical method, for instance a magnetic resonance recording, with a temporal resolution which is suited to determining perfusion and/or diffusion information. With the aid of the image recordings of the second method, perfusion and/or diffusion information is determined for at least one part of the period of measurement and the positron emission measurement information is evaluated as a function of the perfusion and/or diffusion information.

This approach is successful for many regions but is however unsuitable for recordings of the brain. The reason behind this is the particular property of the blood-brain barrier, which actively transports or selectively allows certain substances to pass and selectively blocks others. For instance, the magnetic resonance contrast agents usually used are generally gadolinium chelates (e.g. Gd-DPTA), in other words small molecules, and the PET tracers are for instance a fluorodeoxyglucose (FDG) or a fluorothymidine (FLT), which exhibit very different behavior at the blood-brain barrier. The gadolinium chelates do not pass directly across the blood-brain barrier, if this functions properly, whereas FDG passes very easily across the blood-brain barrier for instance.

### SUMMARY

In at least one embodiment of the present invention, a method is provided which enables a function or malfunction of the blood-brain barrier and an absorption or metabolism of a substance, in particular a PET tracer, in the tissue to be assessed at the same time. Since a specific accumulation of a substance, for instance a PET tracer, generally takes place in the tissue only in very small concentrations, for instance nanomolar and less to the point of few molecules per cell, whereas conventional magnetic resonance contrast agents must be present in much higher concentrations so as to be able to generate an evaluatable signal. In at least one embodiment, the absorption or metabolism of the PET tracer in the tissue is assessed reliably under these circumstances.

According to at least one embodiment of the present invention, a method is disclosed for a nuclear medicine examination of a patient. According to at least one embodiment of the present invention, a use of a fluorodeoxyglucose with a fluorine 19 isotope as a magnetic resonance contrast agent and a fluorodeoxyglucose with a fluorine 18 isotope as a nuclear medicine tracer during an imaging examination is disclosed. According to at least one embodiment of the present invention, the use of a chelate of diethylenetriaminepentaacetic acid with gadolinium as a magnetic resonance contrast agent and of a chelate of diethylenetriaminepentaacetic acid with technetium as a nuclear medicine tracer during an imaging examination is disclosed. According to at least one embodiment of the present invention, the use of radioactively marked iron oxide nanoparticles as magnetic resonance contrast agent and nuclear medicine tracer during an imaging examination is disclosed. According to at least one embodiment of the present invention, the use of a gadolinium chelate as magnetic resonance contrast agent and nuclear medicine tracer during an imaging examination is disclosed. According to at least one embodiment of the present invention, a system is disclosed. According to at least one embodiment of the present invention, a computer program product is disclosed. According to at least one embodiment of the present invention, an electronically readable data carrier is disclosed. The dependent claims define example and advantageous embodiments of the invention.

According to an embodiment of the present invention, a method is provided for a nuclear medicine examination of a patient. With an embodiment of the method, a magnetic resonance recording of an examination region of the patient is created, after a magnetic resonance contrast agent has been administered to the patient. A distribution, for instance a perfusion or a diffusion, of the magnetic resonance contrast agent in the examination region is automatically determined from the magnetic resonance recording.

Furthermore, a nuclear medicine recording of the examination region of the patient is created after a nuclear medicine tracer has been administered to the patient. The magnetic resonance contrast agent and the nuclear medicine tracer have essentially identical pharmacokinetic properties. The nuclear medicine recording is corrected as a function of the distribution of the magnetic resonance contrast agent in the examination region. The nuclear medicine recording may be a positron emission tomography recording (PET) for instance and the nuclear medicine tracer accordingly a PET tracer.

According to a further embodiment, metal-organic chelates are used as magnetic resonance contrast agents and nuclear medicine tracers. The chelates have pharmacokinetically and chemically similar properties, but may feature different properties depending on characteristics relating to detection physics. For instance, a chelate of diethylenetriaminepentaacetic acid with gadolinium (Gd-DTPA) can be used as a magnetic resonance contrast agent and a chelate of diethylenetriaminepentaacetic acid with technetium (99Tc-DTPA) can be used as a nuclear medicine tracer, in particular as a SPECT tracer for a single proton emission tomography recording.

According to a further embodiment, particles are used which show a good contrast effect during magnetic resonance tomography and can be easily radioactively marked, for instance by the integration or adhesion of radioactive substances to the particles. The particles may include iron oxide nanoparticles for instance which are radioactively marked for instance with technetium, fluorine or rubidium. Furthermore, the particles can be functionalized, i.e. they can be provided with specific binding points for cell receptors. The use of functionalized particles of this type is particularly advantageous for the method, since these particles can barely overcome a healthy blood-brain barrier.

According to a further embodiment, a radioactive gadolinium chelate, for instance a chelate of diethylenetriaminepentaacetic acid with a gadolinium 153 isotope (153Gd-DTPA) is used as a magnetic resonance contrast agent and as a nuclear medicine tracer. The radioactive gadolinium chelate is chemically identical to currently standard magnetic resonance contrast agents. The long physical half life of the isotope does not result in an increased radiation exposure of the patient since the biological half life is very short.

According to an embodiment of the present invention, a system is also provided for a nuclear medicine examination of a patient. The system includes a magnetic resonance tomograph, a positron emission tomograph and a control facility. The control facility is able to create a magnetic resonance recording of an examination region of the patient after a magnetic resonance contrast agent has been administered to the patient. Furthermore, the control facility is able to determine a distribution, for instance a perfusion or a diffusion, of the magnetic resonance contrast agent in the examination region from the magnetic resonance recording.

Furthermore, the control facility is configured to create a nuclear medicine recording of the examination region of the patient after a nuclear medicine tracer has been administered to the patient and to correct the nuclear medicine recording as a function of the distribution of the magnetic resonance contrast agent in the examination region. The magnetic resonance contrast agent and the nuclear medicine tracer have essentially identical pharmacokinetic properties.

The system can also be configured such that it is suited to implementing the afore-described method or one of its embodiments. The advantages of the inventive system therefore essentially correspond to the advantages of the afore-described inventive method so that there is no need to repeat the description of the advantages here.

Furthermore, in accordance with an embodiment of the present invention, a computer program product, in particular a computer program or software, which can be loaded directly into a memory of a programmable control facility of a positron emission tomography magnetic resonance system, is provided. All or various of the afore-described embodiments of the inventive method can be implemented with this computer program product, when the computer program product runs in the control facility. The computer program product here optionally requires program segments, e.g. libraries and auxiliary functions so as to realize the corresponding embodiments of the method.

In other words, the claim which focuses on the computer program product is intended to protect in particular a computer program or software, with which one of the afore-described embodiments of the inventive method can be implemented and/or which executes the embodiment. The software may be a source code (e.g. C++ or Java), which is post-compiled (translated) and bound or which only has to be interpreted or an executable software code, which only has to be loaded into the corresponding computing unit for execution purposes.

Finally, an embodiment of the present invention provides an electronically readable data carrier, e.g. a DVD, CD, magnetic tape or USB stick, on which electronically readable control information, in particular software, is stored. When this control information (software) is read by the data carrier and stored in a control facility of a positron emission tomography magnetic resonance system, all the inventive embodiments of the previously described method can be implemented.

### BRIEF DESCRIPTION OF THE DRAWINGS

The present invention is described in detail below with the aid of example embodiments with reference to the figures.

FIG. 1 shows a schematic representation of a mode of operation of a nuclear medicine tracer and a magnetic resonance contrast agent according to an embodiment of the present invention.

FIG. 2 shows a program flow chart of an inventive method.

FIG. 3 shows a schematic representation of an inventive positron emission tomography magnetic resonance system.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

According to an embodiment of the present invention, a combined magnetic resonance examination and nuclear medicine examination, for instance positron emission tomography (PET), is implemented. Here a magnetic resonance contrast agent and a nuclear medicine tracer, for instance a PET tracer, are used, which have identical or similar pharmacokinetic properties, specifically during the transfer across the blood-brain barrier. For instance, substances can be selected, which belong to the same substance class and have a similar polarity or a similar molecular weight. In particular, a chemically identical substance can also be used for the magnetic resonance contrast agent and the PET tracer.

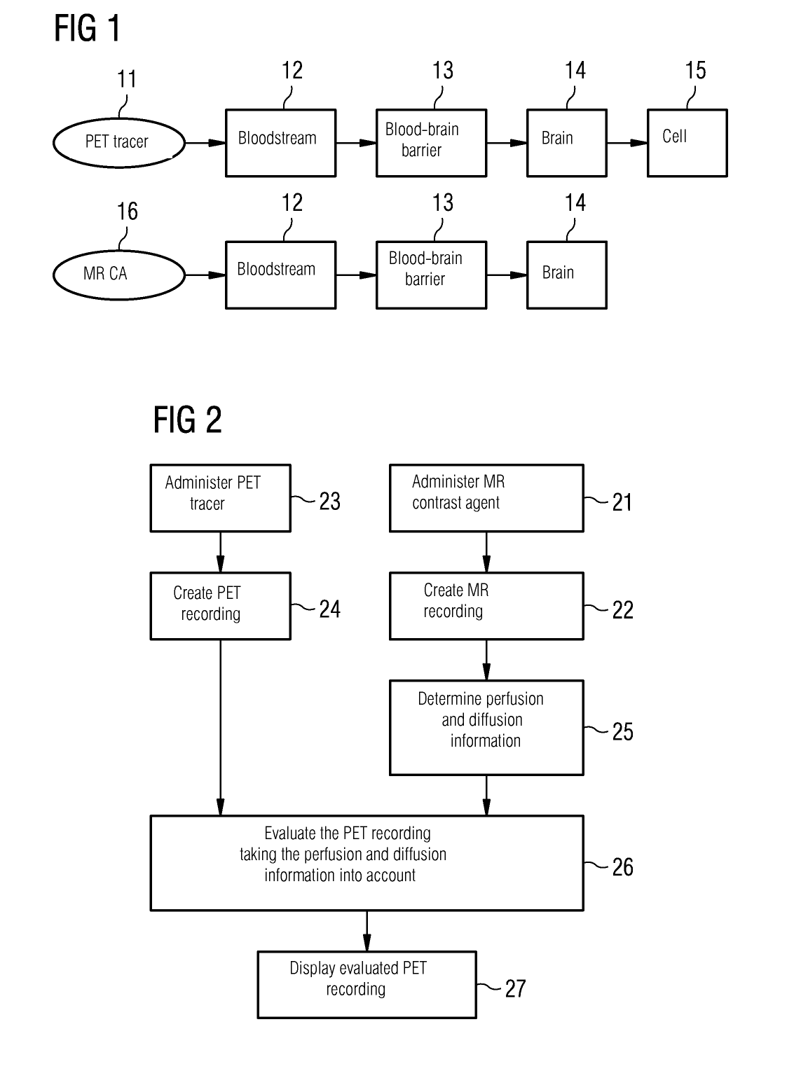

FIG. 1 shows a schematic representation of the mode of operation of a PET tracer 11 and a magnetic resonance contrast agent 16 with identical or similar pharmacokinetic properties.

The PET tracer 11 is injected into the bloodstream 12 of a patient. The PET tracer 11 is distributed in the body of the patient via the bloodstream 12 and overcomes the blood-brain barrier 13 of the patient and thus also reaches the brain 14 of the patient. The PET tracer 11 is deposited on cells 15 of a lesion, for instance a brain tumor, in the brain 14 of the patient or is absorbed by these cells 15 or accumulates therein. The PET tracer 11 can be detected in the cell 15 with the aid of positron emission tomography.

The magnetic resonance contrast agent 16, which comprises similar or identical pharmcokinetic properties to the PET tracer 11, is likewise injected into the bloodstream 12 of the patient and likewise overcomes the blood-brain barrier 13 of the patient to the same degree as the PET tracer 11 on account of the identical pharmacokinetic properties and thus reaches the brain 14 of the patient. The magnetic resonance contrast agent 16 can be detected there with the aid of magnetic resonance tomography. The magnetic resonance contrast agent 16 may also be able to be absorbed by the cell 15 in the brain, but this property of the magnetic resonance contrast agent 16 is not necessary for the method of an embodiment of the present invention.

An implementation of the inventive method is described in detail below with reference to FIG. 2. A magnetic resonance examination is implemented, i.e. a magnetic resonance recording is created (step 22), relatively soon after a magnetic resonance contrast agent has been administered (step 21). The creation of the magnetic resonance recording (step 22) can take place 60 seconds to 15 minutes after the magnetic resonance contrast agent has been administered (step 21) for instance since the magnetic resonance contrast agent shows a good distribution in the tissue in this time and is not yet eliminated. A nuclear medicine examination, for instance a PET or SPECT examination (step 24), takes place for instance 10 minutes to 60 minutes after a corresponding PET tracer and/or SPECT tracer has been administered (step 23), so that the nuclear medicine tracer can accumulate in the tissue. To be able to implement a simultaneous or quasi-simultaneous magnetic resonance and nuclear medicine examination so as to rule out a displacement of the organs between the examinations for instance, the nuclear medicine tracer is administered first and the magnetic resonance contrast agent correspondingly later.

When administering a combined agent comprising a magnetic resonance contrast agent and a nuclear medicine tracer, for instance a mixture of 18F-DG and 19F-DG, the magnetic resonance examination can either take place first or a suitable time frame can be selected in which the suitable measurement times of both methods overlap, for instance 10 to 15 minutes after the injection. If perfusion is disrupted, for instance if the patient has had a stroke for instance, an early magnetic resonance recording can also be prepared during the perfusion phase, for instance 15 to 60 seconds after the injection so as to detect perfusion.

The following substances can be used for instance as magnetic resonance contrast agent and nuclear medicine tracers:
Fluorodeoxyglucose (FDG), which can be detected with a fluorine 19 isotope (19F-DG) during magnetic resonance tomography by measuring at fluorine frequencies instead of proton frequencies, and which can be detected with a fluorine 18 isotope (18F-DG) during positron emission tomography;
Metal-organic chelates, for instance a chelate of diethylenetriaminepentaacetic acid with gadolinium (Gd-DTPA) as a magnetic resonance contrast agent and a chelate of diethylenetriaminepentaacetic acid with a technetium 99 isotope (99Tc-DTPA) as a SPECT tracer;
particles which show a good contrast effect during magnetic resonance tomography and can be easily radioactively marked, for instance iron oxide nanoparticles, e.g. Resovist®, which can be radioactively marked for instance with technetium, fluorine or rubidium; or a radioactive gadolinium chelate, e.g. a chelate of diethylenetriaminepentaacetic acid with a gadolinium 153 isotope (153Gd-DTPA) as both a magnetic resonance contrast agent and also a nuclear medicine tracer.

A magnetic resonance recording and a PET recording are available as a result of steps 22 and 24. The magnetic resonance recording shows the transfer of the magnetic resonance contrast agent across the blood-brain barrier, on the basis of which perfusion and diffusion information relating to the magnetic resonance contrast agent can be determined (Step 25). The PET recording shows a specific binding of the nuclear medicine tracer to cells or regions, in particular lesions, like for instance a brain tumor.

The information from the magnetic resonance recording and the nuclear medicine recording are analyzed together (step 26) so as to be able to assess the information from the nuclear medicine recording. For instance, a mask can be created from the magnetic resonance recording in step 26, said mask being placed over the PET recording. All regions, which do not show magnetic resonance contrast agent accumulation, are identified accordingly in the PET recording, being marked with color for instance, so that a reporting physician knows that no transfer of the PET tracer across the blood-brain barrier can be expected in these regions and the PET examination in this region therefore has no diagnostic significance. The PET recording thus evaluated is displayed on a monitor for instance for analysis by the reporting physician (step 27).

Alternatively, a pharmacokinetic modeling of the binding of the PET tracer can also be implemented in step 26. The concentration of the PET tracer in the extracellular space can be estimated from the magnetic resonance recording as a function of the signal amplification by the corresponding magnetic resonance contrast agent. The quantity of PET tracer bound to receptors of the cells is known as a function of the radiation emitted at this point from the PET recording. The density of the receptors, which bind the PET tracer, can be calculated from the concentration and the bound quantity (step 26) and can be displayed graphically for a reporting physician (step 27).

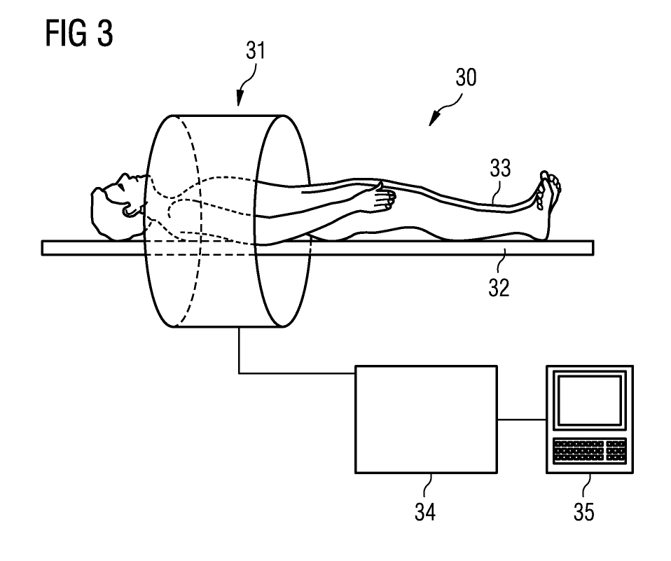

FIG. 3 shows a schematic representation of a system 30, which is suited to implementing an embodiment of the afore-described inventive method. The system 30 includes a measuring facility 31, for instance a combined magnetic resonance and positron emission tomograph, which enables a recording of positron emission measurement information and magnetic resonance information. The patient 33 arranged on a patient couch 32 is moved into the measuring facility 31 in order to implement the measurement. PET recordings and magnetic resonance recordings are created in the measuring facility 31, as previously described, after administering a PET tracer and a magnetic resonance contrast agent with identical or similar pharmacokinetic properties. In this way the PET recordings provide details of functional processes in the body of the patient 33, in particular in the brain of the patient 33, while perfusion and diffusion information are obtained from the magnetic resonance recordings together with additional structure information.

The information, which is recorded in the measuring facility 31, is forwarded to a control facility 34, which derives on the one hand perfusion and diffusion data and on the other hand PET recordings from the information, with this data being analyzed together. The magnetic resonance recordings can be used for instance to trigger a temporal profile of the perfusion and diffusion of the magnetic resonance contrast agent in the body of the patient 33. The evaluated measurement information of the measuring facility 31 is then shown as an image on an image display device 35.

According to an embodiment of the present invention, a method is provided for a nuclear medicine examination of a patient. With an embodiment of the method, a magnetic resonance recording of an examination region of the patient is created, after a magnetic resonance contrast agent has been administered to the patient. A distribution, for instance a perfusion or a diffusion, of the magnetic resonance contrast agent in the examination region is automatically determined from the magnetic resonance recording.

Furthermore, a nuclear medicine recording of the examination region of the patient is created after a nuclear medicine tracer has been administered to the patient. The magnetic resonance contrast agent and the nuclear medicine tracer have essentially identical pharmacokinetic properties. The nuclear medicine recording is corrected as a function of the distribution of the magnetic resonance contrast agent in the examination region. The nuclear medicine recording may be a positron emission tomography recording (PET) for instance and the nuclear medicine tracer accordingly a PET tracer.

Since the magnetic resonance contrast agent and the nuclear medicine tracer have essentially identical pharmacokinetic properties, the distribution, perfusion or diffusion of the nuclear medicine tracer will be essentially identical to that of the magnetic resonance contrast agent in the examination region so that it is possible to conclude the distribution of the nuclear medicine tracer from the distribution of the magnetic resonance contrast agent.

The examination region may include part of the brain of the patient for instance. The pharmacokinetic properties of the magnetic resonance contrast agent and of the nuclear medicine tracer may be essentially identical in respect of overcoming the blood-brain barrier of the patient for instance and are administered outside of the brain. The distribution of the magnetic resonance contrast agent can be automatically determined in the magnetic resonance recording and on account of the same pharmacokinetic properties of the magnetic resonance contrast agent and of the nuclear medicine tracer, it is possible to conclude therefrom a distribution of the nuclear medicine tracer in the brain of the patient.

Furthermore, the pharmacokinetic properties of the magnetic resonance contrast agent and of the nuclear medicine tracer can also be pharmacokinetically identical in respect of an absorption or accumulation in a sub-region of the examination region or in respect of absorption in the bloodstream of the patient, distribution in the examination region, metabolism in a tissue of the examination region or degradation in the examination region. As a result, an absorption or metabolism of the nuclear medicine tracer can be assessed in a tissue in the brain irrespective of a function or malfunction of the blood-brain barrier, even if the specific accumulation of the PET tracer in the tissue has a considerably lower concentration compared with the magnetic resonance contrast agent. The mixing ratio of magnetic resonance contrast agent to nuclear medicine tracer can be 10⁵:1 or greater for instance. As a result, the distribution of the magnetic resonance contrast agent in the examination region can be reliably determined and minimal radiation exposure of the patient can be ensured at the same time.

According to an embodiment, the nuclear medicine recording is corrected by determining regions in the magnetic resonance recording which only show a minimal or even no accumulation of the magnetic resonance contrast agent and marking these regions in the magnetic resonance recording and the nuclear medicine recording. A reporting physician, for instance a doctor, can then identify with the aid of the marked regions that no transfer of the nuclear medicine tracer across the blood-brain barrier is to be expected in these regions and the nuclear medicine recording in these regions therefore has no diagnostic significance.

According to a further embodiment, the correction of the nuclear medicine recording includes determining a concentration of the nuclear medicine tracer in an extracellular region as a function of a signal amplification by the magnetic resonance contrast agent in the magnetic resonance recording. Furthermore, a quantity of nuclear medicine tracer which is bound to receptors is determined with the aid of the nuclear medicine recording and a density of the receptors, which bind the nuclear medicine tracer, is determined as a function of the concentration of the nuclear medicine tracer and of the quantity of nuclear medicine tracer which is bound to the receptors. The binding of the nuclear medicine tracer to cell receptors is thus modeled on the basis of the pharmacokinetically identical properties of the magnetic resonance contrast agent and of the nuclear medicine tracer.

Since the concentration of the nuclear medicine tracer in the extracellular space can be estimated from the magnetic resonance recording as a function of the signal amplification by the corresponding magnetic resonance contrast agent, and the quantity of nuclear medicine tracer which is bound to the receptors is known as a function of the radiation emitted at this point, the density of the receptors, which bind the nuclear medicine tracer, can be calculated and displayed graphically for instance.

According to a further embodiment, the nuclear medicine recording may be a single proton emission tomography recording (SPECT) and the nuclear medicine tracer may accordingly be a SPECT tracer.

According to a further embodiment, the magnetic resonance contrast agent and the nuclear medicine tracer may belong to an identical substance class or have a similar polarity or a similar molecular weight. As a result, the magnetic resonance contrast agent and the nuclear medicine tracer can have essentially identical pharmcokinetic properties.

Substances with pharmacokinetically identical or very similar properties may be substances for instance which are chemically identical or similar, but are different with respect to the detection physics, i.e. in particular with respect to their detectability during magnetic resonance tomography and detectability during positron emission tomography. One example of a substance of this type is fluorodeoxyglucose (FDG), which can be detected as fluorodeoxyglucose with a fluorine 19 isotope (19F-DG) during magnetic resonance tomography by measuring at fluorine frequencies instead of proton frequencies, and which can be detected as fluorodeoxyglucose with a fluorine 18 isotope (18F-DG) during positron emission tomography. Both isotopes are chemically identical and therefore have identical pharmacokinetic properties.

According to an embodiment, a mixture of 19F-DG and 18F-DG can be given, which can then be detected with both modalities, i.e. during both magnetic resonance tomography and also during positron emission tomography. The mixture here can consist primarily of 19F-DG so as to minimize the radiation exposure for the patient as a result of the 18F-DG and to enable magnetic resonance detectability. The mixing ratio of 19F-DG to 18F-DG may be for instance 10⁵ to 1 or greater, typically 10⁶ to 10⁸ to 1.

According to a further embodiment, metal-organic chelates are used as magnetic resonance contrast agents and nuclear medicine tracers. The chelates have pharmacokinetically and chemically similar properties, but may feature different properties depending on characteristics relating to detection physics. For instance, a chelate of diethylenetriaminepentaacetic acid with gadolinium (Gd-DTPA) can be used as a magnetic resonance contrast agent and a chelate of diethylenetriaminepentaacetic acid with technetium (99Tc-DTPA) can be used as a nuclear medicine tracer, in particular as a SPECT tracer for a single proton emission tomography recording.

According to a further embodiment, particles are used which show a good contrast effect during magnetic resonance tomography and can be easily radioactively marked, for instance by the integration or adhesion of radioactive substances to the particles. The particles may include iron oxide nanoparticles for instance which are radioactively marked for instance with technetium, fluorine or rubidium. Furthermore, the particles can be functionalized, i.e. they can be provided with specific binding points for cell receptors. The use of functionalized particles of this type is particularly advantageous for the method, since these particles can barely overcome a healthy blood-brain barrier.

According to a further embodiment, a radioactive gadolinium chelate, for instance a chelate of diethylenetriaminepentaacetic acid with a gadolinium 153 isotope (153Gd-DTPA) is used as a magnetic resonance contrast agent and as a nuclear medicine tracer. The radioactive gadolinium chelate is chemically identical to currently standard magnetic resonance contrast agents. The long physical half life of the isotope does not result in an increased radiation exposure of the patient since the biological half life is very short.

According to an embodiment of the present invention, a system is also provided for a nuclear medicine examination of a patient. The system includes a magnetic resonance tomograph, a positron emission tomograph and a control facility. The control facility is able to create a magnetic resonance recording of an examination region of the patient after a magnetic resonance contrast agent has been administered to the patient. Furthermore, the control facility is able to determine a distribution, for instance a perfusion or a diffusion, of the magnetic resonance contrast agent in the examination region from the magnetic resonance recording.

Furthermore, the control facility is configured to create a nuclear medicine recording of the examination region of the patient after a nuclear medicine tracer has been administered to the patient and to correct the nuclear medicine recording as a function of the distribution of the magnetic resonance contrast agent in the examination region. The magnetic resonance contrast agent and the nuclear medicine tracer have essentially identical pharmacokinetic properties.

The system can also be configured such that it is suited to implementing the afore-described method or one of its embodiments. The advantages of the inventive system therefore essentially correspond to the advantages of the afore-described inventive method so that there is no need to repeat the description of the advantages here.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for a nuclear medicine examination of a patient, the method comprising:
creating a magnetic resonance recording of an examination region of the patient, after a magnetic resonance contrast agent has been administered to the patient;
automatically determining a distribution of the magnetic resonance contrast agent in the examination region from the magnetic resonance recording;
creating a nuclear medicine recording of the examination region of the patient, after a nuclear medicine tracer has been administered to the patient, with the magnetic resonance contrast agent and the nuclear medicine tracer including essentially identical pharmacokinetic properties; and
correcting the nuclear medicine recording as a function of the distribution of the magnetic resonance contrast agent in the examination region.

2. The method as claimed in claim 1, wherein the correction of the nuclear medicine recording comprises:
determining regions in the magnetic resonance recording, which do not show any accumulation of the magnetic resonance contrast agent; and
marking the regions in at least one of the magnetic resonance recording and the nuclear medicine recording.

3. The method as claimed in claim 1, wherein the correction of the nuclear medicine recording comprises:
determining a concentration of the nuclear medicine tracer in an extracellular region as a function of a signal amplification by the magnetic resonance contrast agent in the magnetic resonance recording;
determining a quantity of the nuclear medicine tracer bound to receptors by way of the nuclear medicine recording; and
determining a density of the receptors, which bind the nuclear medicine tracer, as a function of the concentration of the nuclear medicine tracer and the quantity of nuclear medicine tracer bound to the receptors.

4. The method as claimed in claim 1, wherein the examination region comprises at least part of the brain of the patient, and wherein the magnetic resonance contrast agent and the nuclear medicine tracer have been administered outside the brain.

5. The method as claimed in claim 1, wherein the pharmacokinetic properties of the magnetic resonance contrast agent and of the nuclear medicine tracer are essentially identical with respect to overcoming the blood-brain barrier of the patient.

6. The method as claimed in claim 1, wherein the pharmacokinetic properties of the magnetic resonance contrast agent and of the nuclear medicine tracer are essentially identical with respect to at least one of absorption and accumulation in at least one sub-region of the examination region of the patient.

7. The method as claimed in claim 1, wherein the pharmacokinetic properties comprise at least one of
absorption into the bloodstream of the patient,
distribution in the examination region,
metabolism in a tissue in the examination region, and
degradation in the examination region.

8. The method as claimed in claim 1, wherein the nuclear medicine recording is a positron emission tomography recording and the nuclear medicine tracer is a PET Tracer.

9. The method as claimed in claim 1, wherein the nuclear medicine recording is a single photon emission tomography recording and the nuclear medicine tracer is a SPECT tracer.

10. The method as claimed in claim 1, wherein the magnetic resonance contrast agent and the nuclear medicine tracer at least one of
belong to the same substance class,
have a similar polarity and
have a similar molecular weight.

11. The method as claimed in claim 1, wherein the magnetic resonance contrast agent is a fluorodeoxyglucose with a fluorine 19 isotope and the nuclear medicine tracer comprises a fluorodeoxyglucose with a fluorine 18 isotope.

12. The method as claimed in claim 1, wherein the magnetic resonance contrast agent and the nuclear medicine tracer are administered as a mixture.

13. The method as claimed in claim 1, wherein the mixing ratio of magnetic resonance contrast agent and nuclear medicine tracer is greater than 10⁵ to 1.

14. The method as claimed in claim 1, wherein the magnetic resonance contrast agent and the nuclear medicine tracer comprise metal-organic chelates.

15. The method as claimed in claim 1, wherein the magnetic resonance contrast agent comprises a chelate of diethylenetriaminepentaacetic acid with gadolinium and the nuclear medicine tracer comprises a chelate of diethylenetriaminepentaacetic acid with technetium.

16. The method as claimed in claim 1, wherein the magnetic resonance contrast agent and the nuclear medicine tracer comprise particles which are radioactively marked.

17. The method as claimed in claim 16, wherein the particles comprise iron oxide nanoparticles.

18. The method as claimed in claim 16, wherein the particles are radioactively marked with at least one of technetium, fluorine and rubidium.

19. The method as claimed in claim 16, wherein the particles are provided with binding points for cell receptors.

20. The method as claimed in claim 1, wherein the magnetic resonance contrast agent and the nuclear medicine tracer comprise a gadolinium chelate.

21. The method as claimed in claim 20, wherein the gadolinium chelate comprises a gadolinium 153 isotope.

22. A method, comprising:
using a fluorodeoxyglucose with a fluorine 19 isotope as a magnetic resonance contrast agent and using a fluorodeoxyglucose with a fluorine 18 isotope as a nuclear medicine tracer during an imaging examination of a patient, wherein, during the examination
a magnetic resonance recording of an examination region of the patient is created after the magnetic resonance contrast agent has been administered to the patient,
a distribution of the magnetic resonance contrast agent in the examination region is determined from the magnetic resonance recording,
a nuclear medicine recording of the examination region of the patient is created after the nuclear medicine tracer has been administered to the patient, and
the nuclear medicine recording is corrected as a function of the distribution of the magnetic resonance contrast agent in the examination region.

23. The method as claimed in claim 22, wherein the fluorodeoxyglucose with the fluorine 19 isotope and the fluorodeoxyglucose with the fluorine 18 isotope are used as the mixture.

24. The method as claimed in claim 23, wherein the mixing ratio of fluorodeoxyglucose with the fluorine 19 isotope and fluorodeoxyglucose with the fluorine 18 isotope is greater than 10⁵ to 1.

25. A method, comprising:
using a chelate of diethylenetriaminepentaacetic acid with gadolinium as a magnetic resonance contrast agent and using a chelate of diethylenetriaminepentaacetic acid with technetium as a nuclear medicine tracer during an imaging examination of a patient, wherein, during the examination
a magnetic resonance recording of an examination region of the patient is created after the magnetic resonance contrast agent has been administered to the patient,
a distribution of the magnetic resonance contrast agent in the examination region is determined from the magnetic resonance recording,
a nuclear medicine recording of the examination region of the patient is created after the nuclear medicine tracer has been administered to the patient, and
the nuclear medicine recording is corrected as a function of the distribution of the magnetic resonance contrast agent in the examination region.

26. A method, comprising:
using iron oxide nanoparticles, which are radioactively marked, as magnetic resonance contrast agent and nuclear medicine tracer during an imaging examination of a patient, wherein, during the examination
a magnetic resonance recording of an examination region of the patient is created, after the iron oxide nanoparticles have been administered to the patient,
a distribution of the iron oxide nanoparticles in the examination region is determined from the magnetic resonance recording,
a nuclear medicine recording of the examination region of the patient is created, and
the nuclear medicine recording is corrected as a function of the distribution of the iron oxide nanoparticles in the examination region.

27. The method as claimed in claim 26, wherein the particles are radioactively marked with technetium, fluorine and or rubidium.

28. The method as claimed in claim 26, wherein the particles are provided with binding points for cell receptors.

29. A method, comprising:
using a gadolinium chelate as magnetic resonance contrast agent and nuclear medicine tracer during an imaging examination of a patient, wherein, during the examination
a magnetic resonance recording of an examination region of the patient is created after the gadolinium chelate has been administered to the patient,
a distribution of the gadolinium chelate in the examination region is determined from the magnetic resonance recording,
a nuclear medicine recording of the examination region of the patient is created, and
the nuclear medicine recording is corrected as a function of the distribution of the gadolinium chelate in the examination region.

30. The method as claimed in claim 29, wherein the gadolinium chelate comprises a gadolinium 153 isotope.

31. A system comprising:
a magnetic resonance tomograph;
a positron emission tomograph; and
a control facility, wherein the control facility is configured,
to create a magnetic resonance recording of an examination region of a patient, after a magnetic resonance contrast agent has been administered to the patient,
to determine a distribution of the magnetic resonance contrast agent in the examination region from the magnetic resonance recording,
to create a nuclear medicine recording of the examination region of the patient after a nuclear medicine tracer has been administered to the patient, with the magnetic resonance contrast agent and the nuclear medicine tracer having essentially identical pharmacokinetic properties, and
to correct the nuclear medicine recording as a function of the distribution of the magnetic resonance contrast agent in the examination region.

32. The system as claimed in claim 31, wherein the system is configured to
create a magnetic resonance recording of an examination region of the patient, after a magnetic resonance contrast agent has been administered to the patient;
automatically determine a distribution of the magnetic resonance contrast agent in the examination region from the magnetic resonance recording;
create a nuclear medicine recording of the examination region of the patient, after a nuclear medicine tracer has been administered to the patient, with the magnetic resonance contrast agent and the nuclear medicine tracer including essentially identical pharmacokinetic properties; and
correct the nuclear medicine recording as a function of the distribution of the magnetic resonance contrast agent in the examination region.

33. A computer program product, loadable directly into a memory of a programmable control facility of a positron emission tomography magnetic resonance system, comprising program segments, so as to execute all the steps of the method as claimed in claim 1, when the program is executed in the control facility.

34. An electronically readable data carrier with electronically readable control information stored thereupon, configured to implement the method as claimed in claim 1 when the data carrier is used in a control facility of a positron emission tomography magnetic resonance system.

35. The method as claimed in claim 2, wherein the correction of the nuclear medicine recording comprises:
determining a concentration of the nuclear medicine tracer in an extracellular region as a function of a signal amplification by the magnetic resonance contrast agent in the magnetic resonance recording;
determining a quantity of the nuclear medicine tracer bound to receptors by way of the nuclear medicine recording; and
determining a density of the receptors, which bind the nuclear medicine tracer, as a function of the concentration of the nuclear medicine tracer and the quantity of nuclear medicine tracer bound to the receptors.

36. The method as claimed in claim 17, wherein the particles are radioactively marked with at least one of technetium, fluorine and rubidium.

37. The method as claimed in claim 17, wherein the particles are provided with binding points for cell receptors.

38. The method as claimed in claim 27, wherein the particles are provided with binding points for cell receptors.

39. A tangible computer readable medium including program segments for, when executed on a computer device, causing the computer device to implement the method of claim 1.
